(19) United States
(12) Patent Application Publication
(10) Pub. No.: US 2014/0155754 A1
(43) Pub. Date: Jun. 5, 2014

(54) HAIR TREATMENT DEVICE WITH HAIR DETECTOR

(71) Applicants: Babu Varghese, Eindhoven (NL); Rieko Verhagen, Vught (NL); Natallia Eduardauna Uzunbajakava, Eindhoven (NL)

(72) Inventors: Babu Varghese, Eindhoven (NL); Rieko Verhagen, Vught (NL); Natallia Eduardauna Uzunbajakava, Eindhoven (NL)

(73) Assignee: KONINKLIJKE PHILIPS N.V., EINDHOVEN (NL)

(21) Appl. No.: 14/236,412

(22) PCT Filed: Aug. 8, 2012
(86) PCT No.: PCT/IB2012/054033

§ 371 Date: Jan. 31, 2014

Related U.S. Application Data
(60) Provisional application No. 61/525,866, filed on Aug. 22, 2011.

(51) Int. Cl. A61B 18/20; A61B 5/00

(52) U.S. Cl. CPC A61B 18/203; A61B 5/0059
USPC 600/473; 606/9

(57) ABSTRACT

The invention provides a hair treatment device (40) comprising a light-based detector (10) for detecting a hair (22) near a skin surface (21) and a method for detecting a hair (22) near a skin surface (21). The detector (10) comprises a light source (11), optical elements (14, 16, 17, 18) and a polarization-sensitive light sensor (12, 13). The light source (11) generates a light beam (31) and the optical elements (14, 16, 17, 18) focus the light beam (31) at a hair (22) near the skin surface (21). The polarization-sensitive light sensor (12, 13) is provided for detecting light interacted with the hair (22) or the skin surface (21) and having a predefined linear polarization. The light source (11) and/or the optical elements (14, 16, 17, 8) are arranged to cause the light beam (31), when reaching the skin surface (21), to have a polarization direction which is time-invariant and spatially variant in cross-sections of the 10 light beam.

### FIELD OF THE INVENTION

This invention relates to a hair treatment device comprising a light-based detector for detecting a hair near a skin surface, the detector comprising a light source for generating a light beam, optical elements for focusing the light beam at a hair near the skin surface and a polarization-sensitive light sensor for detecting light interacted with the hair or the skin surface and having a predefined linear polarization.

This invention further relates to a shaving device with a light-based detector as described herebefore and to a method of detecting hairs near a skin surface.

### BACKGROUND OF THE INVENTION

Such a hair treatment device is, e.g., known from the US patent application published as US 2010/0063491 A1. This patent application describes a detector for detecting a hair near a skin surface of a body part. The device comprises a light source and a sensor for detecting radiation returning from said hair. The device further comprises an elliptical, preferably circular, polarizer between the source and said skin surface for providing elliptically or circularly polarized light at the skin surface. Light reflection or scattering at an air-skin interface does not significantly change the direction of polarization. In light reflected or scattered at a hair the polarization direction is changed due to cortex birefringence and scattering by cortex and medulla. Optical elements including a polarizing beam splitter cause the sensor not to detect the light reflected at the air-skin interface.

The detector of US 2010/0063491 A1 has been developed to solve a problem of other prior art optical hair detectors using linearly polarized light. In detectors using linearly polarized light, the reliability of the detection depends on the orientation of the hair relative to the direction of polarization. The use of circularly polarized light makes the detector of US 2010/0063491 A1 more or less independent of the orientation of the hair, which renders the detection more reliable. However, also when circularly polarized light is used, the contrast is not yet completely independent of hair orientation. One of the problems of this known device is that the light beam may (partly) lose its elliptical or circular polarization before it reaches the skin.

### OBJECT OF THE INVENTION

In view of the above, it is an object of the invention to provide a hair treatment device with a light-based detector for detecting a hair near a skin surface, using alternative measures to make the detection independent of the hair orientation.

### SUMMARY OF THE INVENTION

According to a first aspect of the invention, this object is achieved by providing a hair treatment device comprising a light-based detector for detecting a hair near a skin surface. The detector comprises a light source for generating a light beam, optical elements for focusing the light beam at a hair near the skin surface, and a polarization-sensitive light sensor for detecting light interacted with the hair or the skin surface and having a predefined linear polarization. The light source and/or the optical elements are arranged to cause the light beam, when reaching the hair or the skin surface, to have a polarization direction which is time-invariant and spatially variant in cross-sections of the light beam.

In a light beam with a time-invariant spatially variant polarization direction in cross-sections of the light beam, the direction of the polarization vector is different for different positions in the beam cross section and does not vary over time. This is different from linearly polarized light wherein the polarization vector has a similar direction in every position in the beam cross section, and also different from non-polarized light which does not have a defined polarization direction at all and wherein the polarization direction changes randomly over time. It is also different from circularly polarized light wherein the polarization direction in every position in the beam cross section changes during the polarization period.

Two well known examples of spatially variant polarization are radial polarization and azimuthal polarization. In radially polarized light, in every position in the beam cross section the polarization vector points towards or away from the center of the beam cross-section (see FIG. 1a). In azimuthally polarized light, the polarization vector is tangential to the center of the beam cross-section in any position in the beam cross-section (see FIG. 1b). In the following, the invention will be discussed using radially polarized light, but the invention works in a similar way for azimuthally polarized light or light with other types of spatially variant polarization.

When the light interacts with the hair or the skin surface, the light may, e.g., be scattered, reflected or refracted. In addition, depending on the skin surface structure, the polarization state of the beam may or may not change. When the light beam hits the human skin surface, the light maintains its polarization. If the light hits the surface at a position where a hair is located, the polarization will change (see FIG. 2 for an example). Because the polarization in some directions changes more than the polarization in other directions, the beam loses its radial polarization. The sensor sensitive to light with a predefined direction of polarization detects the reflected light. If the light beam does have a dominant direction of polarization, then the sensor is preferably sensitive to light with a different direction of polarization. When the polarization state of the reflected beam differs from the original space variant polarization of the incident beam, the measured intensity of the light detected by the polarization sensitive sensor is also changed. This effect is independent of the orientation of the hair. Although the orientation of the hair affects the polarization profile of the reflected beam, the intensity of the beam at the predefined polarization direction always differs from the intensity that would be measured for an unaffected beam. The use of the time-invariant spatially variant polarized incident light beam thus provides a reliable detection method that does not depend on the orientation of the hairs to be detected.

The time-invariant spatially variant polarization may, e.g., be obtained using a light source that is operative to produce the light beam with the time-invariant spatially variant polarization. This can, e.g., be obtained by providing the laser source with a conical Brewster prism. Another way of providing the incident light beam with the desired polarization may be to place a spatially varying retarder in the optical path between the laser source and the surface to be scanned. Alternatively, the optical elements may comprise an LCoS chip, designed to transform the polarization state of the incident light beam to a radial, azimuthal or other type of spatially variant polarization.

In order to further improve the accuracy of the hair detection, the device may further comprise a second polarization-sensitive sensor for detecting light reflected, scattered or refracted at the hair or the skin surface and having a polarization orthogonal to the predefined linear polarization. When the polarization state of the light beam is preserved, the sensor aligned with the polarization of the incoming light will measure maximum intensity or, in the event that the polarization state of the light beam is depolarized, both sensors will measure similar intensities. When the polarization state of the light beam is changed by a hair, the different sensors will measure different intensities. A difference between or ratio of the intensities measured by both sensors is a good indication of the presence of a hair.

The hair treatment device according to the invention may further be adapted for cutting or removing the detected hair. Such a shaving device has the advantage that, due to the accurate hair detection, it will only attempt to cut or remove the actual hairs. This reduces the possible detrimental effects that the cutting or removing mechanism may have on the skin.

According to a further aspect of the invention, a method for detecting a hair near a skin surface is provided. The method comprises generating a light beam, focusing the light beam at a hair near the skin surface, and detecting light interacted with the hair or the skin surface and having a predefined linear polarization. The generating and/or the focusing cause the light beam, when reaching the hair or the skin surface, to have a polarization direction which is time-invariant and spatially variant in cross-sections of the light beam.

These and other aspects of the invention are apparent from and will be elucidated with reference to the embodiments described hereinafter.

### BRIEF DESCRIPTION OF THE DRAWINGS

In the drawings:

FIG. 1a shows a cross-section of a light beam with a radial polarization,

FIG. 1b shows a cross-section of a light beam with an azimuthal polarization,

FIG. 2a shows a cross-section of a light beam with a radial polarization after interacting with a hair,

FIG. 2b shows a cross-section of a light beam with an azimuthal polarization after interacting with a hair,

FIG. 3 shows a light-based detector according to the invention,

FIG. 4 shows a spatially varying retarder, and

FIG. 5 shows a shaving device according to the invention.

### DETAILED DESCRIPTION OF THE INVENTION

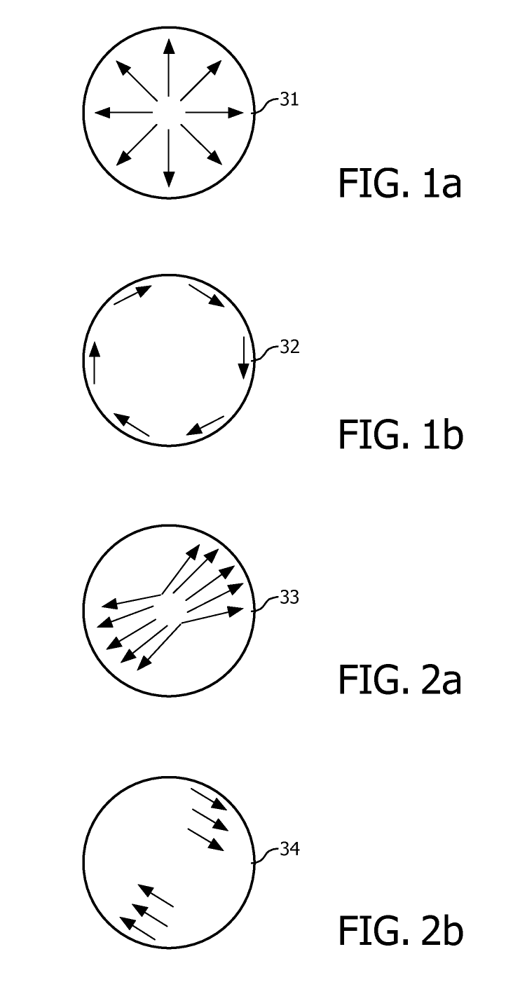

FIG. 1a shows a cross-section of a light beam 31 with a radial polarization. At every position in the cross-section of the beam 31, the polarization points towards or away from the center of the cross-section of the beam 31. FIG. 1b shows a cross-section of a light beam 32 with an azimuthal polarization. At every position in the cross-section of the light beam 32, the polarization points tangentially relative to center of the light beam 32. When the radially or azimuthally polarized incident light beams 31, 32 hit, e.g., a skin surface, the light beams 31, 32 are reflected without changing the directions of polarization.

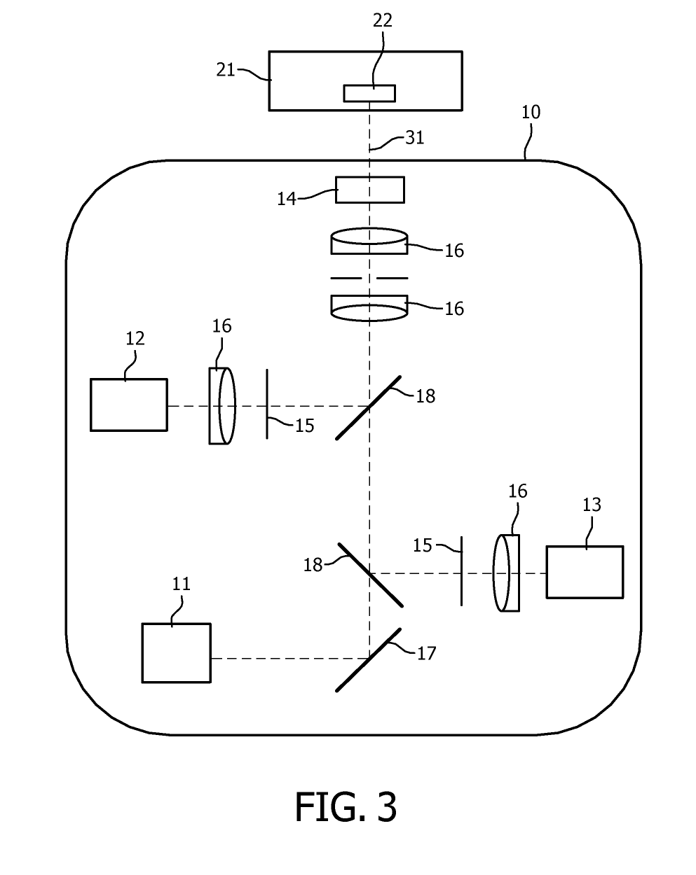

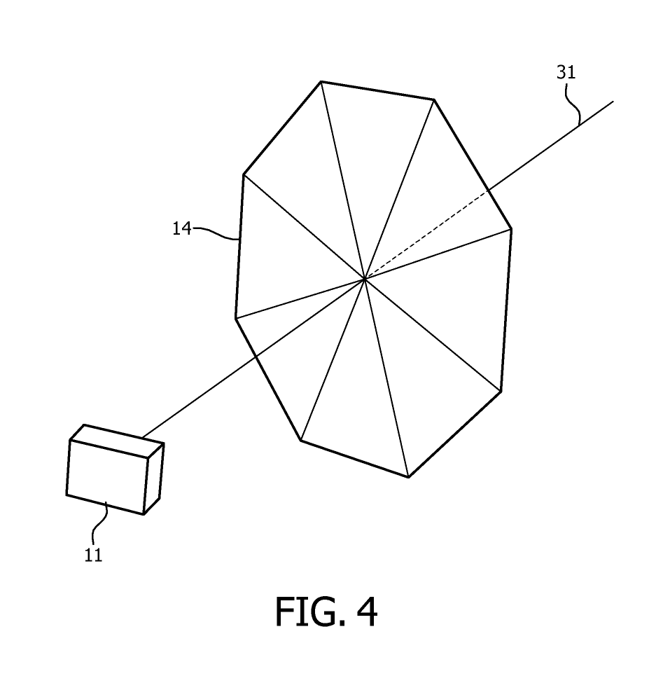

FIG. 2a shows a cross-section of a light beam 33 after interaction with a hair. When the radially polarized light beam 31 of FIG. 1a interacts with the hair, the light beam 31 is reflected and its polarization direction is changed. The hair does however not change all polarization directions to the same extent. Some polarization vectors are rotated over a relatively large angle, while other polarization vectors are rotated over smaller angles or are hardly rotated at all. A schematic example of the polarization state of a cross-section of the reflected light beam 33 is shown in FIG. 2a. The exact polarization state of the reflected light beam 33 depends on the polarization state of the incoming light beam 31 and the surface texture and orientation of the hair. As will be elucidated below, with reference to FIGS. 3 and 4, this polarization rotating effect can be used for detecting hairs. FIG. 2b shows a cross-section of a light beam 34 with an azimuthal polarization after refraction at a hair. When the azimuthally polarized light beam 32 of FIG. 1b interacts with a hair, the beam 32 is reflected and its polarization direction is changed.

FIG. 3 shows a light-based detector 10 according to the invention. The detector 10 according to the invention is adapted to detect hairs 22 on human or animal skin 21. Hair detection may be useful in, for example, IPL (Intense Pulsed Light) based or laser based shaving, hair removing or hair-growth reduction apparatuses. Alternatively, the detector 10 may be used in other types of hair treatment devices, e.g. hair colouring devices. The light-based detector 10 of FIG. 3 comprises a laser source 11 for emitting a laser beam, preferably in the near-infrared or infrared part of the spectrum. For example, light with a wavelength of 785 or 850 nm may be used. Optical elements, like lenses 16 and/or mirrors 17 focus the light beam on the skin 21. A control unit (not shown) coupled to the laser source 11 and/or (part of) the optical elements 16, 17 controls the exact optical path of the laser beam in order to control the exact area of skin 21 that is tested for the presence of a hair 22 and to enable scanning lines or 2D areas of skin 21.

According to the invention, the light beam 31 incident on the hair or skin 21 has a time-invariant and spatially variant polarization direction in cross-sections of the light beam 31. The light beam 31 may, e.g., have a radial or azimuthal polarization. In the following, radially polarized light will be used to describe the invention, but the same detector 10 may also be used with azimuthally polarized light or other types of time-invariant spatially variant polarized light. The radial polarization may be obtained in different ways. For example, the laser source 11 itself may provide the light with the radial polarization. This may be obtained by providing the laser source 11 with a conical Brewster prism.

Another way of providing the incident light beam 31 with the radial polarization may be to place a spatially varying retarder 14 in the optical path between the laser source 11 and the skin surface 21 to be scanned. A spatially varying retarder 14 converts linearly polarized light into a radial or near-radial polarization distribution. For providing the spatially varying retarder 14 with linearly polarized light, the laser source 11 itself may provide linearly polarized light or a polarizer should be positioned somewhere in the light path for providing linearly polarized light to the spatially varying retarder 14. An example of a spatially varying retarder 14 will be discussed below with reference to FIG. 4. Alternatively, the optical elements may comprise an LCoS chip, designed to transform the polarization state of the incident light beam to a radial or azimuthal polarization.

The incident light beam 31 with radial polarization is reflected by a hair 22 or the skin 21. If, at the position of reflection, no hair is present, the reflected light beam has the same or a similar polarization state as the incoming light beam 31 (see FIG. 1a). If, however, the incident light beam 31 hits upon a hair 22, the polarization state of the beam will change. Because the polarization in some directions changes more than the polarization in other directions, the beam loses its radial or near-radial polarization state. FIG. 2a shows an example of the polarization state of the light beam after reflection at the hair 22.

The reflected beam then re-enters the detector 10. The optical elements 16, 17 lead the returning beam to a polarization-sensitive light sensor 12. Semi-transparent mirrors 18 may be used for providing different light paths for the outgoing and the returning light beams. The light sensor 12 is sensitive to light with a predefined direction of polarization. This may be achieved by providing a polarizing filter 15, which only lets through light with a specific polarization direction. Preferably, the polarizing filter 15 is configured such that the light sensor 12 gives a minimum response during a calibration measurement with a non-birefringent material. When calibrated this way, any measured signal above said minimum response indicates an increased probability of having detected a hair 22 as a birefringent object. If the incident light beam has a dominant polarization direction, this may be achieved by making the light sensor 12 sensitive to light with a polarization orthogonal to the dominant incident polarization.

Optionally, a second polarization-sensitive light sensor 13 may be provided for detecting light with a polarization direction which is orthogonal to that of the light detected by the first light sensor 12. The control unit may, e.g., use a difference between or ratio of both signals to provide a measure for the probability that a hair is detected.

FIG. 4 shows a spatially varying retarder 14. This spatially varying retarder 14 is composed of eight sectors of λ/2 wave plates to create a time-invariant spatially variant polarization and for transforming linearly polarized light into light having a near-radial polarization distribution. Each sector rotates the polarization vector of the incoming linearly polarized light beam to a different angle. Experiments have shown that the polarization distribution in the near field just after passing through the spatially varying retarder 14 with eight sectors is close to a perfectly radial polarization distribution, except for the air gaps between the sectors. Similar results will be obtained when using a different number of sectors.

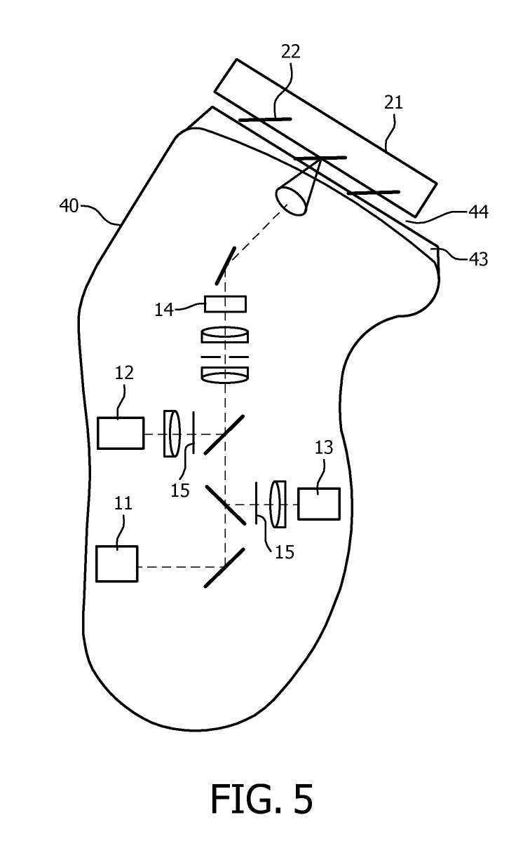

FIG. 5 shows a shaving device 40 according to the invention. The shaving device 40 comprises a hair detector similar to the one described above with reference to FIG. 3. Equal reference numbers correspond to similar features. In addition to features already discussed above, the shaving device 40 may also comprise an optical or contact window 43 and an immersion fluid 44 for improving the penetration properties of the radiation into the skin 21. For example, the fluid 84 may be an index matching fluid, having an index of refraction which is halfway between that of the optical window and that of the skin 21. Preferably, all refractive indices are substantially equal. This also lowers the reflection from the skin 21. The fluid 44 may also be selected for the purpose of cooling the skin 21, or treating it otherwise. Furthermore, although the contact window 43 is optional, it helps in serving as a reference for determining positions of skin objects, such as the hairs 22.

The shaving device 40 may not only use the laser source 11 for detecting the hair 22, but also for cutting it. When the laser source 11 is used for cutting, it may operate at a different power level than when detecting hairs 22. Alternatively, a separate laser source (not shown) is used for the cutting of the hairs 22. The control over the cutting process may be performed by the control unit or by an additional cutting processor (not shown). The cutting processor is coupled to the light-based detector 10 to activate the hair-cutting laser source in a focal position of the hair-cutting laser beam near the skin surface 21 in which the light-based detector has detected the presence of a hair 22.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A hair treatment device comprising a light-based detector for detecting a hair near a skin surface, the detector comprising:
a light source for generating a light beam,
optical elements for focusing the light beam at the hair near the skin surface, and
a polarization-sensitive light sensor for detecting light interacted with the hair or the skin surface and having a predefined linear polarization,
wherein the light source and/or the optical elements are arranged to cause the light beam, when reaching the hair or the skin surface, to have a polarization direction which is time-invariant and spatially variant in cross-sections of the light beam.

2. A hair treatment device as claimed in claim 1, wherein the light source and/or the optical elements are arranged to cause the light beam to have a radial or an azimuthal polarization direction in cross-sections of the light beam.

3. A hair treatment device as claimed in claim 1, wherein the light source is operative to produce a light beam having a polarization direction which is time-invariant and spatially variant in cross-sections of the light beam.

4. A hair treatment device as claimed in claim 3, wherein the light source comprises a conical Brewster prism.

5. A hair treatment device as claimed in claim 1, wherein the optical elements comprise a spatially varying retarder.

6. A hair treatment device as claimed in claim 1, wherein the optical elements comprise an LCoS chip.

7. A hair treatment device as claimed in claim 1, further comprising a second polarization-sensitive sensor for detecting light interacted with the hair or the skin surface and having a polarization orthogonal to the predefined linear polarization.

8. A hair treatment device according to claim 1, the device further comprising a hair-cutting laser source for generating a hair-cutting laser beam and a processor which is coupled to the light-based detector, wherein the processor is arranged to activate the hair-cutting laser source in a focal position of the hair-cutting laser beam near the skin surface in which the light-based detector has detected the presence of the hair.

9. A method for detecting a hair near a skin surface, the method comprising:
generating a light beam,
focusing the light beam at a hair near the skin surface, and
detecting light interacted with the hair or the skin surface and having a predefined linear polarization,
wherein the generating and/or the focusing cause the light beam, when reaching the hair or the skin surface, to have a polarization direction which is time-invariant and spatially variant in cross-sections of the light beam.
